(19) United States
(12) Patent Application Publication
(10) Pub. No.: US 2011/0064816 A1
(43) Pub. Date: Mar. 17, 2011

(54) ATORVASTATIN COMPOSITIONS

(75) Inventors: Alagarsamy Alagumurugan, Tuticorin District (IN); Rakesh Kumar Bhasin, Ghaziabad (IN); Harshal Prabhakar Bhagwatwar, Hyderabad (IN); Rizwan Zafar, District Kishanganj (IN); Saptarshi Nath, Kharagpur (IN); Ranadeep Paramananda Bokalial, Guwahati (IN); Lakshmi Prasanna Gubbala, Secunderabad (IN); Venkata Nookaraju Sreedharala, Hyderabad (IN); Amarjit Maharaj Giri, Hyderabad (IN); Pradeep Jairao Karatgi, Hyderabad (IN); Sanjay Chhagan Wagh, Hyderabad (IN); Ravi Pillai, Hyderabad (IN); Kaushik Atul, Ghaziabad (IN); Varghese Bency, Alleppey (IN)

(73) Assignees: DR. REDDY'S LABORATORIES LTD., Hyderabad, 500 016, Andhra Pradesh (IN); DR. REDDY'S LABORATORIES, INC., Bridgewater, NJ (US)

(21) Appl. No.: 12/992,340

(22) PCT Filed: May 13, 2009
(86) PCT No.: PCT/US2009/043721

§ 371 Date: Nov. 12, 2010

Related U.S. Application Data
(60) Provisional application No. 61/101,514, filed on Sep. 30, 2008.
(60) Provisional application No. 61/101,508, filed on Sep. 30, 2008.
(60) Provisional application No. 61/103,972, filed on Oct. 9, 2008.
(60) Provisional application No. 61/162,746, filed on Mar. 24, 2009.

(30) Foreign Application Priority Data
May 13, 2008 (IN) 1169/CHE/2008
May 13, 2008 (IN) 1170/CHE/2008
Aug. 19, 2008 (IN) 2017/CHE/2008
Jan. 15, 2009 (IN) 99/CHE/2009

(51) Int. Cl. A61K 9/14; A61K 31/40; A61K 9/00; A61P 9/10

(52) U.S. Cl. 424/489; 514/423; 424/400

(57) ABSTRACT

Compositions containing atorvastatin, including its pharmaceutically acceptable salts, solvates, hydrates, enantiomers, polymorphs and their mixtures, and processes for preparing the same. Further aspects relate to pharmaceutical formulations comprising compositions containing atorvastatin, or a salt thereof, processes for preparing the same, and their methods of use, treatment and administration.

### INTRODUCTION

Aspects of the present invention relate to compositions containing atorvastatin, including its pharmaceutically acceptable salts, solvates, hydrates, enantiomers, polymorphs and their mixtures, and processes for preparing the same. Further aspects relate to pharmaceutical formulations comprising compositions containing atorvastatin, or a salt thereof, processes for preparing the same, and their methods of use, treatment and administration.

Embodiments of the present invention relate to pharmaceutical formulations comprising compositions containing atorvastatin, or a salt thereof, and at least one alkali metal salt in a concentration range of about 1% to about 75% by weight of the formulation.

Atorvastatin calcium, a potent molecule from the “statin” family, is a lipid-lowering agent that acts by inhibiting the HMG-CoA reductase enzyme. Atorvastatin calcium has a chemical name [R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid, calcium salt (2:1) trihydrate and has structural Formula I.

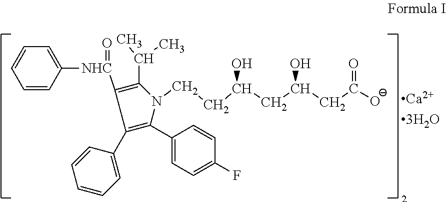

A commercially available product containing atorvastatin is LIPITOR® oral tablets, distributed by Pfizer. LIPITOR® tablets contain atorvastatin as its calcium salt trihydrate and are available in 10, 20, 40 and 80 mg atorvastatin acid equivalent strengths. LIPITOR® is indicated for prevention of cardiovascular diseases and hypercholesterolemia.

Atorvastatin calcium is a white to off-white crystalline powder that is insoluble in aqueous solutions of pH 4 and below. Atorvastatin calcium is very slightly soluble in distilled water, pH 7.4 phosphate buffer and acetonitrile, slightly soluble in ethanol, and freely soluble in methanol. Atorvastatin calcium is susceptible to degradation when exposed to heat, moisture, light and low pH conditions, which is primarily due to the degradation from the carboxylic form to a lactone form.

U.S. Patent Application Publication No. 2003/0175338 discloses a pharmaceutical composition of atorvastatin calcium and other salts, for example atorvastatin magnesium, etc., having particle sizes less than 150 μm, such composition having improved bioavailability. U.S. Patent Application Publication No. 2005/0032880 discloses a composition of amorphous atorvastatin calcium, wherein the amorphous atorvastatin is layered over a core.

It poses a challenge for the scientists to formulate a new dosage forms comprising atorvastatin or its salts which exhibit an improved stability or at least a comparative stability to a commercial product such as LIPITOR tablets and which are similar in properties to the commercial formulation under in vivo and/or in vitro conditions.

### SUMMARY

An aspect of the present invention includes pharmaceutical compositions, comprising atorvastatin, or a pharmaceutically acceptable salt thereof, combined with at least one surfactant and acid-soluble polymer, and optionally other excipients.

An aspect of the present invention includes pharmaceutical compositions comprising atorvastatin or a pharmaceutically acceptable salt thereof, combined with at least one surfactant and at least one alkaline metal salt, and optionally other excipients.

An aspect of the present invention includes pharmaceutical compositions, comprising nanoparticulate atorvastatin, or a pharmaceutically acceptable salt thereof, and one or more pharmaceutically acceptable excipients.

An aspect of the present invention includes pharmaceutical compositions, comprising:

(a) atorvastatin or a pharmaceutically acceptable salt thereof in one layer;

(b) at least one alkaline compound in another layer; and

(c) one or more pharmaceutically acceptable excipients in either or both layers.

An embodiment of the aspect of the present invention includes pharmaceutical compositions, comprising:

(a) atorvastatin or a pharmaceutically acceptable salt thereof in one layer;

(b) at least one alkali metal salt and/or alkaline earth metal salt in another layer; and

(c) one or more pharmaceutically acceptable excipients in either or both layers.

An aspect of the present invention relates to pharmaceutical formulations comprising compositions containing atorvastatin or its salts, and at least one alkali metal salt in a concentration range of about 1% to about 75% by weight of the formulation.

An aspect of the present invention relates to processes for preparation of pharmaceutical compositions containing atorvastatin or its salts and formulations comprising such compositions.

An aspect of the present invention relates to methods of use, prevention, treatment and administration of the pharmaceutical formulations comprising compositions containing atorvastatin or its salts according to the present invention.

### DETAILED DESCRIPTION

Atorvastatin and its salts can readily be prepared as described, for example, in U.S. Pat. Nos. 4,681,893, 5,273,995, and 5,969,156, which are incorporated herein by reference.

Aspects of the present invention relate to pharmaceutical compositions comprising atorvastatin or its pharmaceutically acceptable salts, solvates, hydrates, enantiomers, polymorphs or their mixtures, processes for preparing the same, and their methods of use, treatment and administration.

Embodiments of the present invention include solubility-enhanced forms of atorvastatin or its salts.

In an embodiment, the present invention includes pharmaceutical compositions and/or formulations comprising solubility-enhanced forms of atorvastatin or its salts.

The various pharmaceutically acceptable salts of atorvastatin or its salts include metal salts and amine salts. Pharmaceutically acceptable metal salts include, but are not limited to, sodium, potassium, lithium, calcium, magnesium, aluminum, iron, and zinc salts. Such salts may be derived from bases such as sodium hydroxide, potassium hydroxide, lithium hydroxide, calcium hydroxide, magnesium hydroxide, zinc hydroxide, aluminum hydroxide, ferrous or ferric hydroxide, and ammonium hydroxide. Pharmaceutically acceptable amine salts include, but are not limited to, salts formed by reaction with ammonium hydroxide or organic amines such as for example methylglucamine, choline, arginine, 1-deoxy-2-(methylamino)-D-glucitol, and the like.

The amount of the active ingredient in pharmaceutical compositions of the present invention will be a therapeutically effective amount. Generally, a therapeutically effective amount ranges from about 0.05% to about 70%, or from about 1% to about 60%, or from about 5% to about 50%, by weight, based on the total weight of the pharmaceutical composition.

The term “solubility-enhanced form” as used in the present invention relates to atorvastatin or its salts wherein particle sizes are less than about 2000 nm, or compositions comprising atorvastatin or its salts wherein particle sizes are less than about 2000 nm. Solubility-enhanced forms of atorvastatin or its salts may be in liquid form like suspensions or in solid form such as powders, granules or pellets. Solubility-enhanced forms may further comprise at least one surface stabilizer.

The term “surface stabilizer” as used in the context of the present invention relates to any agent, which chemically affects particle surfaces and enhances the solubility of atorvastatin or its salts.

The term “micronized” used in the context of the present invention relates to compositions comprising atorvastatin or its salts whose particle size is greater than about 2000 nm.

The term “nanoparticulate” as used in the context of the present invention relates to particles of atorvastatin or its salts where the particle sizes are less than 2000 nm.

The term “atorvastatin” as used in the context of the present invention relates to the acid form, a salt form, a polymorphic crystalline or amorphous form or mixtures of such forms, solvates, ethers, esters, etc.

The term “composition” as used in the context of the present invention includes solubility-enhanced forms, micronized forms, and nanoparticulate forms of atorvastatin that exhibit an improved dissolution or solubility, and/or improved stability, compared to atorvastatin alone. The compositions may be in the forms of multiparticulates such as powders, granules, pellets, spheroids, extrudates, minitablets, and the like.

The term “formulation” as used in context of the present invention includes pharmaceutical dosage forms such as tablets, capsules, pills, sachets, etc.

As atorvastatin is an insoluble molecule, the compositions comprising atorvastatin suffer from the problem of poor solubility resulting in a relatively low bioavailability.

Nanoparticulate compositions of a poorly soluble therapeutic agent have been described in U.S. Pat. No. 5,145,684. Nanoparticulate compositions can offer one or more of the following advantages: (1) faster onset of action; (2) an increased rate of dissolution; and (3) increased bioavailability; thereby improving performance characteristics suitable for oral administration. The absolute bioavailability of atorvastatin reportedly is approximately 14%, and this is attributed to pre-systemic clearance in gastrointestinal mucosa and/or hepatic first pass metabolism.

In an embodiment the invention includes pharmaceutical compositions and/or formulations comprising solubility-enhanced forms of atorvastatin or its salts, wherein bioavailability of atorvastatin is improved.

In the present invention, the value for a particle size distribution value for D₅₀ of a powder is the particle size below which 50% of the atorvastatin particles fall. Similarly, D₁₀ and D₉₀ are the particle sizes below which 10% and 90%, respectively, of the particles fall. Embodiments of the invention include pharmaceutical formulations that utilize atorvastatin, or a salt thereof, wherein a D₉₀ value is less than about 1500 nm, less than about 1000 nm, less than about 750 nm, less than about 500 nm, or less than about 250 nm. Further embodiments include pharmaceutical formulations that utilize atorvastatin, or a salt thereof, wherein a D₅₀ value is less than about 750 nm, less than about 500 nm, less than about 250 nm, or less than about 125 nm.

Solid pharmaceutical formulations typically do not permit analysis of particle sizes for the included drug, so the particle size specifications are to be interpreted as applying to the ingredient used to prepare the formulations.

In another embodiment the invention includes solubility-enhanced forms comprising atorvastatin or its salts and at least one surface stabilizer.

Useful surface stabilizers which can be employed in the invention include, but are not limited to, various organic and inorganic pharmaceutical excipients. Such excipients include various polymers, low molecular weight oligomers, natural products, and surfactants. Surface stabilizers include nonionic, cationic, zwitterionic and ionic surfactants. Representative examples of useful surface stabilizers include hydroxypropyl methylcelluloses, hydroxypropylcelluloses, polyvinylpyrrolidones, sodium lauryl sulfate, dioctylsulfosuccinate, gelatin, casein, lecithin (phosphatides), dextran, gum acacia, cholesterol, tragacanth, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tween™ products, e.g., Tween 20 and Tween 800 (ICI Speciality Chemicals)), polyethylene glycols (e.g., Carbowax 3550 and 934 (Union Carbide)), polyoxyethylene stearates, carboxymethylcellulose calcium, carboxymethyl cellulose sodium, methylcelluloses, hydroxyethylcelluloses, hydroxypropyl methylcellulose phthalates, magnesium aluminium silicate, triethanolamine, polyvinyl alcohols (PVA), poloxamers (e.g., Pluronic™ products F68 and F108Q, which are block copolymers of ethylene oxide and propylene oxide); poloxamines (e. g., Tetronic™ 908, also known as poloxamine 908, which is a tetrafunctional block copolymer derived from sequential addition of propylene oxide and ethylene oxide to ethylenediamine (BASF Wyandotte Corporation, Parsippany, N.J.)), Tetronic™ 15080 (T-1508) (BASF Wyandotte Corporation), PEG-derivatized phospholipids, PEG-derivatized cholesterols, PEG-derivatized cholesterol derivatives, PEG-derivatized vitamin A, PEG-derivatized vitamin E, lysozyme, random copolymers of vinyl pyrrolidone and vinyl acetate, and the like.

Nanoparticulate atorvastatin compositions in the form of nanosuspension can be made using process steps including, for example, milling, microfluidization, high pressure homogenization, or precipitation techniques. Following milling, homogenization, precipitation, etc., the resultant nanoparticulate atorvastatin composition can be utilized in solid or liquid dosage formulations, such as controlled release formulations, solid dose fast melt formulations, aerosol formulations, nasal formulations, lyophilized formulations, tablets, capsules, solid lozenge, powders, etc.

Milling atorvastatin to obtain a nanoparticulate dispersion comprises dispersing atorvastatin particles in a liquid dispersion medium in which atorvastatin is poorly soluble, followed by applying mechanical means in the presence of grinding media to reduce the particle size of atorvastatin to the desired effective average particle size. The dispersion media can be, for example, water, safflower oil, ethanol, t-butanol, glycerin, polyethylene glycol (PEG), hexane, or glycol.

Microfluidization is a technique by which samples (suspension based) are fluidized through very tiny orifice resulting in particle size reduction. An intensifier pump pushes a product stream down a channel of fixed geometry and interacts with the interaction chamber with a very high shear. The particles consistently and uniformly collide with the walls and each other, to enhance the size reduction process.

Another method of forming the desired nanoparticulate atorvastatin compositions is by microprecipitation. This is a method of preparing stable dispersions of poorly soluble active agents in the presence of one or more surface stabilizers and one or more colloid stability enhancing surfactants. Such a method comprises, for example: (1) dissolving atorvastatin in a suitable solvent such as methanol, isopropyl alcohol, ethanol, n-butanol etc; (2) adding the solution from step (1) to a solution comprising at least one surface stabilizer; and (3) causing precipitation from step (2) by adding an appropriate anti-solvent, such as water, tetrahydrofuran, etc.

Homogenization can be used to obtain atorvastatin nanoparticulate compositions. High pressure homogenization process involves passing the samples through a tiny orifice under a very high pressure driven by either air or gas. The temperature is controlled throughout the process. An exemplary method comprises dispersing atorvastatin particles in a liquid dispersion medium in which atorvastatin is poorly soluble, followed by subjecting the dispersion to homogenization to reduce the particle size of the atorvastatin to the desired effective average particle size. The dispersion media can be, for example, water, safflower oil, ethanol, t-butanol, glycerin, a hexane, polyethylene glycol (PEG), or another glycol.

In order to process nanosuspensions into the compositions and/or formulations, these nanosuspensions can be converted into solid powder form. Various processes are available to convert these nanosuspensions into solid form, such processes including spray drying, bead layering/layering onto excipients, and lyophilization/freeze-drying.

Spray drying is a commonly used method of drying a liquid feed with heated air. The liquid feed (solution, colloid or suspension) varies depending on the material being dried and transforms the feed from a fluid state into a dried particulate form. The dried form can then be used for further pharmaceutical processes like tableting, etc. It can also be used for drug layering by having a diluent in the spraying media.

Bead layering involves layering the drug solution/suspension onto various beads like microcrystalline cellulose (MCC), sugar, etc. This process involves fluidized bed processor for drug layering and then filling the beads into a suitable capsule, or tabletting, etc.

Lyophilization or freeze-drying is a process of converting the liquid form in to solid form. Freeze drying works by freezing the material and then reducing the surrounding pressure and adding enough heat to allow the frozen water in the material to sublime directly from the solid phase to gas thus leaving the solid material. The freeze-dried or lyophilized material can then be suitably incorporated into solid dosage form.

Atorvastatin calcium is a sensitive molecule and susceptible to heat, moisture, light, low pH, and oxygen, all causing degradation of the molecule. Some of the impurities that form are described below:

1. Atorvastatin lactone (“lactone” impurity) is represented by structural Formula II and has a chemical name (2R trans)-5-(4-fluorophenyl)-2-(1-methyl ethyl)-N,4-diphenyl-1[2-(tetrahydro-4-hydroxy-6-oxo-2H-pyran-2yl)ethyl]-1H-pyrrole-3-carboxamide.

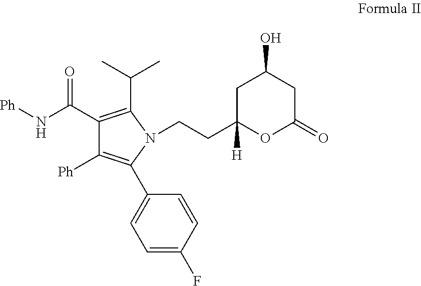

2. A “desfluoro” impurity is represented by structural Formula III and has a chemical name [R-(R*,R*)]-2,3-diphenyl-β,δ,dihydroxy-5-(1-methylethyl)-4-[phenylamino)carbonyl]-1H-pyrrole-1-heptanoic acid, hemi calcium salt.

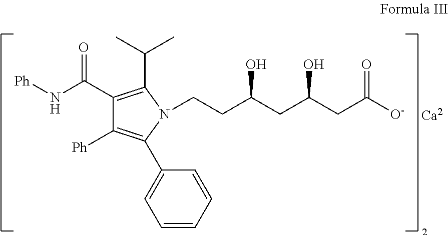

3. An atorvastatin t-butyl ester (“ester” impurity) is represented by structural Formula IV and has a chemical name [R-(R*,R*)]-2-(4-fluorophenyl)-β,δ-dihydroxy-5-[1-methylethyl)-3-phenyl-4-[[(phenylamino)-carbonyl]-1H-pyrrole-1-heptanoic acid, t-butyl ester.

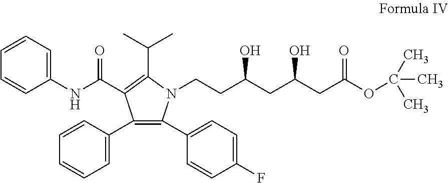

4. An atorvastatin isomer (“isomer” impurity) is represented by structural formula V and has a chemical name [R-(R*,S*)]-2-(4-fluorophenyl)-β,δ,-dihydroxy-5-(1-methylethyl)-3-phenyl-4-[(phenylamino)carbonyl]-1H-pyrrole-1-heptanoic acid, hemi calcium salt.

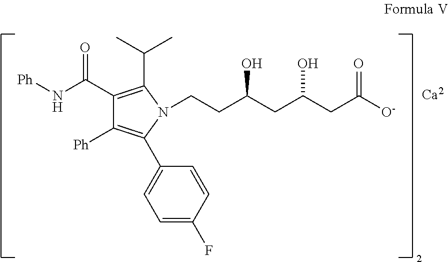

As atorvastatin calcium is susceptible to oxidation, one or more antioxidants can be incorporated into the compositions for its stabilization.

Non-limiting examples of antioxidants that are useful in the present invention include butylated hydroxyanisole (BHA), butylated hydroxytoluene (BHT), citric acid, alpha-tocopherol, gallic acid, and the like, including any mixtures thereof. A combination of atorvastatin calcium and antioxidant may be called an “atorvastatin calcium-antioxidant premix.” For example, “atorvastatin calcium-BHA premix” refers to a combination of atorvastatin calcium and BHA in the context of the present invention. Pre-mixes generally are not simple mixtures of the drug compound and antioxidant, but are intimate mixtures such as solid solutions or other dispersions wherein particles of the components cannot be distinguished using methods such as optical microscopy.

In an embodiment, the present invention provides pharmaceutical compositions comprising (a) atorvastatin or a pharmaceutically acceptable salt thereof in one layer; (b) at least one alkaline compound in another layer; and (c) one or more pharmaceutically acceptable excipients in either or both layers. In an aspect of the embodiment, the alkaline compound is an alkali metal salt and/or analkaline earth metal salt, or mixtures thereof.

In another embodiment the present invention relates to pharmaceutical formulations comprising atorvastatin or its salts, wherein compositions comprising atorvastatin or its salts and compositions comprising alkaline earth metal salts are present as two distinct layers.

In yet another embodiment the invention includes pharmaceutical formulations comprising atorvastatin or its salts wherein compositions comprise an alkalizer other than an alkaline earth metal salt, to provide a stabilizing effect to atorvastatin or its salts.

In embodiments the invention includes pharmaceutical formulations comprising atorvastatin or its salts wherein a composition comprising atorvastatin along with an alkalizer other than an alkaline earth metal salt is present in one layer, and a composition comprising alkaline earth metal salt is present in another layer.

In an embodiment the invention includes pharmaceutical formulations comprising atorvastatin or its salts in the form of multilayered tablets comprising a composition of atorvastatin or its salts and an alkalizer other than an alkaline earth metal salt together in one layer, and a composition comprising an alkaline earth metal salt, wherein the concentration of alkaline earth metal salt is in the range of about 20% to about 99% by weight of the layer composition.

Various alkalizing agents or alkalizers or alkaline compounds that can be used in the present invention include, but are not limited to, inorganic agents like sodium, potassium, magnesium, or calcium salts such as the citrate, carbonate, bicarbonate, phosphate, sulfate, sulfite, benzoate, ascorbate, etc. Organic alkaline compounds such as meglumine are also useful.

Various useful alkaline earth metal salts include but are not limited to calcium chloride, calcium hydroxide, calcium phosphate, calcium phosphate dibasic, calcium phosphate tribasic, calcium citrate, calcium formate, calcium silicate, calcium stearate, calcium sulfate, calcium sulfate dihydrate, calcium sulfate hemihydrate, calcium sulfate anhydrous, calcium acetate, calcium gluconate, calcium ascorbate, calcium lactate, calcium glycinate, calcium citrate, calcium cyclamate, magnesium acetate, magnesium acetylacetonate, magnesium ammonium phosphate, magnesium borocitrate, magnesium chloride, magnesium chlorate, magnesium citrate, magnesium gluconate, magnesium glycerol phosphate, magnesium hydroxide, magnesium salicylate, magnesium sulfate, meglumine, potassium carbonate, potassium bicarbonate, potassium chloride, potassium hydroxide, potassium citrate, potassium metabisulfite, potassium phosphate, sodium carbonate, sodium bicarbonate, sodium bisulphite, sodium bisulphate, sodium chloride, sodium citrate, sodium hydroxide, sodium lactate, sodium phosphate, and any mixtures of two or more thereof.

An embodiment of the present invention is directed to processes for preparing bilayer tablets comprising compositions of atorvastatin or its salts, wherein a blend comprising atorvastatin and a blend comprising an alkalizer are prepared by any of the processes described above and then together compressed to form bilayer tablets.

In an aspect the invention includes processes for preparing pharmaceutical formulations comprising compositions of atorvastatin or its salts, an embodiment of a process comprising:

1) sifting excipients such as diluents, disintegrants, etc. and optionally active ingredient through a sieve and mixing;

2) preparing a granulating solution by dispersing/dissolving a suitable binder in a suitable solvent;

3) granulating step 1) materials with granulating solution of step 2) and drying the granules;

4) optionally, in place of steps 2) and 3), subjecting step 1) materials to roll compaction to form granules;

5) sifting the dried granules of step 3) or granules of step 4) and extragranular excipients through a sieve, and blending;

6) blending a lubricant with the blend of step 5); and

7) compressing the lubricated blend into tablets or filling into capsules.

In an aspect the invention includes pharmaceutical formulations comprising compositions containing:

i) about 1% to about 50% by weight of atorvastatin or its salt;

ii) about 5% to about 75% by weight of an alkalizing agent; and

iii) about 1% to about 50% by weight of a surfactant.

In an embodiment the invention includes pharmaceutical formulations comprising compositions containing atorvastatin or its salt, at least one alkalizing agent and at least one surfactant.

In an embodiment the invention includes pharmaceutical formulations comprising compositions comprising:

i) about 1% to about 50% by weight of atorvastatin or its salt;

ii) about 6% to about 75% by weight of at least one alkalizing agent; and

iii) about 1% to about 50% by weight of at least one surfactant.

In yet another embodiment the invention includes pharmaceutical formulations comprising compositions containing atorvastatin or its salt, wherein weight ratios of alkalizing agent to surfactant are in the range of about 1:0.1 to about 0.1:1.

In an aspect the invention includes processes for preparing pharmaceutical formulations comprising compositions containing atorvastatin or its salts, an embodiment of a process comprising:

1) sifting excipients such as diluents, disintegrants, etc., and optionally active ingredient, through a sieve and mixing;

2) preparing a granulating solution by dispersing/dissolving a suitable binder or surfactant in a suitable solvent;

3) granulating step 1) materials with granulating solution of step 2) and drying the granules;

4) optionally, in place of steps 2) and 3), subjecting step 1) materials to roll compaction to form granules;

5) sifting the dried granules of step 3) or granules of step 4) and extragranular excipients through a sieve, and blending;

6) blending a lubricant with the blend of step 5); and

7) compressing the lubricated blend into tablets or filling into capsules.

In yet another embodiment the invention includes pharmaceutical formulations comprising a composition comprising:

i) atorvastatin or a salt thereof;

ii) one or more surfactants; and

iii) one or more acid solubility-enhancing excipients;

wherein an acid solubility-enhancing excipient is incorporated into intragranular and/or extragranular portions.

In an embodiment the invention includes pharmaceutical formulations comprising compositions containing atorvastatin or a salt thereof, at least one acid soluble polymer and at least one surfactant.

In an embodiment the invention includes pharmaceutical formulations comprising compositions comprising:

i) about 1% to about 50% by weight of atorvastatin or a salt thereof;

ii) about 0.1% to about 50% by weight of at least one acid solubility-enhancing excipient; and

iii) about 1% to about 50% by weight of at least one surfactant.

In embodiments of the present invention, an acid solubility-enhancing excipient comprises a pharmaceutically acceptable polymer that can be water soluble, water swellable, water insoluble, pH dependent, pH independent, or combinations thereof.

Pharmaceutically acceptable polymers in the context of the invention include, but are not limited to, polyethylene glycols (molecular weight ≦about 400), hydroxymethylcelluloses, hydroxyethylcelluloses, hydroxypropylcelluloses, hydroxypropyl methylcelluloses, methylcelluloses, carboxymethylcelluloses (CMC), sodium CMC, carboxyethylcelluloses, carboxy polymethylene, hydroxypropyl methyl phthalate, polyvinylpyrrolidones, cellulose acetates, sodium alginate, gums such as acacia gum, guar gum, tragacanth gum and xanthan gum; methacrylic acid copolymers like poly(butylmethacrylate, (2-dimethylaminoethyl)methacrylate, methylmethacrylate), Eudragit™ products designated E100 or E12.5 or EPO, polyvinyl acetal diethylaminoacetate (available as AEA supplied by Sankyo Co. Limited), chitosan, and the like and mixtures thereof.

Eudragit™ E is a cationic copolymer based on dimethylaminoethyl methacrylate and neutral methacrylates, having solubility in acids and used in pharmaceutical formulations to provide gastro-soluble film coatings that are soluble below about pH 5 and swellable and permeable above about pH 5. The repeating unit in the polymer has the following structure,

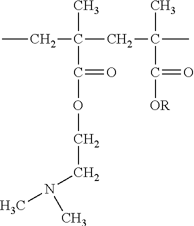

where R represents CH₃ and C₄H₉ groups and the polymer has a molecular weight about 150,000. The Eudragit E100 product is granular, the Eudragit E12.5 product is a 12.5% solution of E100 in isopropanol and acetone, and the Eudragit EPO product is a fine powder made from E100. These products are sold by Evonik Industries AG, Essen, Germany.

In an aspect, the invention includes processes for preparing pharmaceutical formulations comprising compositions containing atorvastatin or its salts, an embodiment of a process comprising:

1) sifting excipients such as diluents, disintegrants, acid solubility-enhancing excipient etc., and optionally active ingredient, through a sieve and mixing;

2) preparing a granulating solution by dispersing/dissolving a suitable binder or surfactant in a suitable solvent;

3) granulating step 1) materials with granulating solution of step 2) and drying the granules;

4) optionally, in place of steps 2) and 3), subjecting step 1) materials to roll compaction to form granules;

5) sifting the dried granules of step 3) or granules of step 4) and extragranular excipients through a sieve, and blending;

6) blending a lubricant with the blend of step 5); and

7) compressing the lubricated blend into tablets or filling into capsules.

In an embodiment the invention includes pharmaceutical formulations containing compositions comprising atorvastatin or its salts. To prepare formulations, various useful pharmaceutically acceptable excipients in the context of the present invention comprise diluents, binders, disintegrants, lubricants, surfactants, glidants, and the like, and optionally coating agents.

Various useful diluents include but are not limited to starches, lactose, mannitol, Pearlitol™ SD 200, cellulose derivatives, confectioner's sugar and the like. Different grades of lactose include but are not limited to lactose monohydrate, lactose DT (direct tableting), lactose anhydrous, Flowlac™ (available from Meggle Products), Pharmatose™ (available from DMV) and others. Different grades of starches include but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that can be used include crystalline celluloses and powdered celluloses. Examples of crystalline cellulose products include but are not limited to CEOLUS™ KG801, Avicel™ PH 101, PH102, PH301, PH302 and PH-F20, microcrystalline cellulose 114, and microcrystalline cellulose 112. Other useful diluents include but are not limited to carmellose; sugar alcohols such as mannitol, sorbitol and xylitol; alkaline earth salts such as calcium carbonate, magnesium carbonate, dibasic calcium phosphate, and tribasic calcium phosphate.

Various useful binders include but are not limited to hydroxypropylcelluloses (Klucel™-LF), hydroxypropyl methylcelluloses or hypromelloses (Methocel™), polyvinylpyrrolidones or povidones (PVP-K25, PVP-K29, PVP-K30, PVP-K90), Plasdone™ S 630 (copovidone), powdered acacia, gelatin, guar gum, carbomers (e.g., Carbopol™), methylcelluloses, polymethacrylates, and starches.

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (FMC-Asahi Chemical Industry Co., Ltd.), crospovidones, examples of commercially available crospovidone products including but not limited to crosslinked povidone, Kollidon™ CL [manufactured by BASF (Germany)], Polyplasdone™ XL, XI-10, and INF-10 [manufactured by ISP Inc. (USA)], and low-substituted hydroxypropylcelluloses. Examples of low-substituted hydroxypropylcelluloses include but are not limited to grades such as LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starches.

Various glidants or antisticking agents include but are not limited to talc, silica derivatives, colloidal silicon dioxide and the like and mixtures thereof.

Various lubricants that can be used include but are not limited to stearic acid and stearic acid derivatives such as magnesium stearate, calcium stearate, zinc stearate, sucrose esters of fatty acid, polyethylene glycol, talc, sodium stearyl fumarate, zinc stearate, castor oils, and waxes.

Surfactants are wetting agents that lower the surface tension of a liquid, allowing easier spreading, and lower the interfacial tension between two liquids. They contain both hydrophobic groups and hydrophilic groups, thus being soluble in both organic solvents and water.

Surfactants may be ionic or nonionic. Ionic surfactants may be anionic, cationic, or zwitterionic. Anionic surfactants include the alkoyl isethionates, alkyl and alkyl ether sulfates and salts thereof, alkyl and alkyl ether phosphates and salts thereof, alkyl methyl taurates, and soaps, such as, for example, alkali metal salts including sodium or potassium salts of long chain fatty acids. Non-limiting examples include chenodeoxycholic acid, 1-octanesulfonic acid sodium salt, sodium deoxycholate, glycodeoxycholic acid sodium salt, N-lauroylsarcosine sodium salt, lithium dodecyl sulfate, sodium cholate hydrate, and sodium lauryl sulfate (SLS), also called sodium dodecyl sulfate (SDS).

Examples of amphoteric and zwitterionic surfactants include but are not limited to carboxy, sulfonate, sulfate, phosphate, and phosphonate compounds. Examples are alkylimino acetates and iminodialkanoates and aminoalkanoates, imidazolinium and ammonium derivatives, betaines, sultaines, hydroxysultaines, alkyl sarcosinates and alkanoyl sarcosinates, and the like.

Nonionic surfactants include polyoxyethylene castor oil derivatives, polyoxyethylene sorbitan fatty acid esters (e.g., the commercially available Tween™ products, e.g., Tween 20 and Tween 800, from ICI Speciality Chemicals); poloxamers (e.g., Pluronic™ products F68 and F108Q, which are block copolymers of ethylene oxide and propylene oxide); poloxamines (e.g., Tetronic™ 908, also known as poloxamine 908, which is a tetrafunctional block copolymer derived from sequential addition of propylene oxide and ethylene oxide to ethylenediamine from BASF Wyandotte Corporation, Parsippany, N.J. USA), and Tetronic™ 15080 (T-1508) (BASF Wyandotte Corporation).

Examples of useful cationic surfactants include, but are not limited to, polymers, biopolymers, polysaccharides, cellulosics, alginates, phospholipids, and nonpolymeric compounds, such as zwitterionic stabilizers, poly-n-methylpyridinium, anthryl pyridinium chloride, cationic phospholipids, chitosan, polylysine, polyvinylimidazole, polybrene, polymethylmethacrylate, polyvinylpyrrolidone-2-dimethylaminoethyl methacrylate, lysozyme, long-chain polymers such as alginic acid, carrageenan (FMC Corp.), and POLYOX™ (Dow Chemical Co., Midland, Mich. USA); cationic lipids, sulfonium, phosphonium, and quarternary ammonium compounds, such as stearyltrimethylammonium chloride, and benzyl-di-(2-chloroethyl)ethylammonium bromide.

Various film-forming agents include but are not limited to cellulose derivatives such as soluble alkyl- or hydroalkyl-cellulose derivatives such as methyl cellulose, hydroxymethylcellulose, hydroxyethyl cellulose, hydroxypropyl cellulose, hydroxymethyethyl cellulose, hydroxypropyl methylcellulose, sodium carboxymethyl cellulose, etc., acidic cellulose derivatives such as cellulose acetate phthalate, cellulose acetate trimellitate and methylhydroxypropylcellulose phthalate, polyvinyl acetate phthalate, etc., insoluble cellulose derivatives such as ethylcellulose and the like, dextrins, starches and starch derivatives, polymers based on carbohydrates and derivatives thereof, natural gums such as gum Arabic, xanthans, alginates, polyacrylic acid, polyvinylalcohols, polyvinyl acetate, polyvinylpyrrolidones, polymethacrylates and derivatives thereof (Eudragit™) chitosan and derivatives thereof, shellac and derivatives thereof, and waxes and fat substances.

In the case of polymethacrylates, cationic copolymerizates of dimethylaminoethyl methacrylate with neutral methacrylic esters (Eudragit™ E), copolymerizates of acrylic and methacrylic esters having a low content of quaternary ammonium groups (described in “Ammonio Methacrylate Copolymer Type A or Type B” USP/NF, Eudragit™ RL and RS, respectively), and copolymerizates of ethyl acrylate and methyl methacrylate with neutral character (in the form of an aqueous dispersion, described in “Polyacrylate Dispersion 30 PerCent” Ph. Eur., Eudragit™ NE 30 D) are useful.

Anionic copolymerizates of methacrylic acid and methyl methacrylate (described in “Methacrylic Acid Copolymer, Type C” USP/NF, Eudragit™ L and S, respectively, or in the form of the Eudragit™ L 30 D aqueous dispersion), acidic cellulose derivatives such as cellulose acetate phthalate, cellulose acetate trimellitate and methylhydroxypropylcellulose phthalate, polyvinyl acetate phthalate, etc. may be used for film coatings.

The coatings may be applied using methods such as film coating, press coating, tablet coating, encapsulating or microencapsulating.

If required, the films may contain additional adjuvants for coating processing such as plasticizers, polishing agents, colorants, pigments, antifoam agents, opacifiers, antisticking agents, and the like.

Various plasticizers include but are not limited to castor oil, diacetylated monoglycerides, dibutyl sebacate, diethyl phthalate, glycerin, polyethylene glycol, propylene glycol, triacetin, and triethyl citrate. Also mixtures of plasticizers may be utilized. The type of plasticizer depends upon the type of coating agent. A plasticizer is frequently present in an amount ranging from about 0.5% (w/w) to about 30% (w/w), based on the total weight of the film coating.

An opacifier like titianium dioxide may also be present in an amount ranging from about 10% to about 20%, based on the total weight of the coating. When colored tablets are desired then the color is normally applied in the coating. Consequently, coloring agents and pigments may be present in the film coating. Various coloring agents include but are not limited to iron oxides, which can be red, yellow, black or blends thereof.

Anti-adhesives are normally used in film coating processes to avoid sticking effects during film formation and drying. An example of an anti-adhesive for this purpose is talc. The anti-adhesive can be present in the film coating in an amount of about 0.5% (w/w) to about 15% (w/w), based upon the total weight of coating.

Suitable polishing agents include polyethylene glycols of various molecular weights or mixtures thereof, talc surfactants (e.g. glycerol mono-stearate and poloxamers), fatty alcohols (e.g., stearyl alcohol, cetyl alcohol, lauryl alcohol and myristyl alcohol) and waxes (e.g., carnauba wax, candelilla wax and white wax). In an embodiment, polyethylene glycols having molecular weights of 3,000-20,000 are employed.

In addition to above the coating ingredients, sometimes pre-formulated coating products such as Opadry™ White OY 58900 (containing hydroxypropyl methylcellulose, PEG 400, and titanium dioxide), Opadry™ AMB White OY-B-28920, Lusterclear™, etc. may be used. Products sold in dry form require only mixing with a liquid before use.

In aspects, the present invention provides methods of use, prevention, treatment and administration of the pharmaceutical formulations comprising compositions containing atorvastatin or its salts. In embodiments, the pharmaceutical formulations of the present invention are useful in the prevention and/or treatment of cardiovascular diseases and hypercholesterolemia.

In embodiments, the pharmaceutical formulations of the present invention comprising atorvastatin or its salts exhibit an improved stability or at least a comparative stability to a commercial product, such as LIPITOR tablets, and exhibit similar properties to the commercial formulation under in vivo and/or in vitro conditions.

Certain specific aspects and embodiments will be further described in the following examples, being provided only for purposes of illustration, and the invention is not to be construed as limited thereto.

### EXAMPLE 1

### Preparation of Atorvastatin Calcium-BHA Pre-Mix

160 g of atorvastatin calcium was added to 1600 mL of ethyl acetate followed by heating to about 65-75° C. to obtain a clear solution, then the solution was cooled to about 25 to 30° C. 0.2 g of butylated hydroxyanisole (“BHA”) was added to the solution, stirred for about 5 to 10 minutes and filtered through a celite bed, followed by washing the bed with 160 mL of ethyl acetate. The filtrate was passed through an agitated thin-film dryer at about 73° C. to 78° C. under a vacuum of about 650 mm Hg. The solid material that was obtained from the agitated thin film dryer was subjected to micronization in a jet mill. The solid material was then dried using a fluid bed dryer at 68° C. to 75° C. for about 4 hours. The resulting atorvastatin calcium-BHA pre-mix was amorphous.

### EXAMPLE 2

### Pharmaceutical Formulation for Atorvastatin 80 mg Tablets

A. Nanoparticle Suspension.

| Ingredient | Quantity | |
|---|---|---|
| Atorvastatin calcium-BHA pre-mix | 30 | g |
| (Example 1) | | |
| Sodium lauryl sulphate | 15 | g |
| Water | 2 | L |

Manufacturing Process:

1) Atorvastatin calcium-BHA pre-mix and sodium lauryl sulphate were placed into a beaker, then 15-20 percent of the total water was added to the beaker and mixed well to obtain a thick paste-like consistency.

2) The remaining amount of water was added to step 1 and the mixture was homogenized using a homogenizer (IKA-WERKF, T18 Ultra-Turrax, Germany), for about 30 minutes at 13,000 rpm until a uniform white suspension formed.

3) The suspension was transferred into a bead mill (Labstar, Netzsch Instruments, UK, with zirconium beads, 0.2-0.3 μm size) and milled for about 90 minutes with feed pump speed ranging from 90-110 rpm and milling speed ranging from about 2400-2550 rpm.

The particle size distribution of suspended particles is below.

| Parameter | Result |
|---|---|
| D₉₀ | 246 nm |
| D₅₀ | 126 nm |
| D₁₀ | 72 nm |

B. Granules.

| Ingredient | Quantity | |
|---|---|---|
| Nanoparticle suspension* | 1.67 | L |
| Microcrystalline cellulose (Avicel ™ PH112) | 122.7 | g |
| Sodium bicarbonate | 9.6 | g |

*5.525 ml of suspension contains 82.87 mg of atorvastatin calcium-BHA pre-mix.
$ Avicel ™ PH112 is a product of FMC Biopolymers.

Manufacturing Process:

1) Microcrystalline cellulose and sodium bicarbonate were co-sifted through an ASTM #30 mesh sieve.

2) Step 1 material was transferred to a fluid bed processor fitted with a top spray assembly and nanoparticle suspension was sprayed onto the material using an inlet temperature of about 70° C. and exhaust temperature of 45° C., spray pump at 10-20 rpm. The formed granules were dried until a loss on drying at 105° C. less than 2.5% by weight was obtained.

C. Tableting.

| Ingredient | Grams |
|---|---|
| Granules | 157.16 |
| Sodium starch glycolate type A | 36 |
| Hydroxypropylcellulose (Klucel ™ LF)* | 4.8 |
| Lactose monohydrate (Flowlac ™ 100)** | 59.42 |
| Microcrystalline cellulose (Avicel PH112) | 39.62 |
| Magnesium stearate | 3 |

*Klucel ™ LF is a product of Aqualon.
**Flowlac ™ 100 is a product of Meggle Pharma.

Manufacturing Process:

1) Drug-containing granules, sodium starch glycolate type A, hydroxypropylcellulose, lactose monohydrate, and microcrystalline cellulose were co-sifted through an ASTM #30 mesh sieve.

2) Step 1) material was uniformly blended in a blender for 15 minutes.

3) Magnesium stearate was sifted through an ASTM #30 mesh sieve, added to step 2), and further uniformly blended for 5 minutes.

4) The final blend of step 3) was compressed into tablets containing 80 mg of atorvastatin.

5) Step 4) tablets were placed into HDPE containers with an oxygen absorbent packet and a desiccant packet, and then the containers were sealed with an induction sealer and stored at 2-8° C. for further use.

The tablets were analyzed and results are shown in Table 1. Disintegration testing was performed according to Test 701 “Disintegration” of United States Pharmacopeia 29, United States Pharmacopeial Convention, Inc., Rockville, Md., 2005 (“USP”).

TABLE 1

| Parameter | Result |
|---|---|
| Content Uniformity, % relative standard | 1.4 |
| deviation | |
| Weight loss at 105° C., percent | 4.91 |
| Disintegration time in water, minutes | <5 |

Impurities (% of label atorvastatin content)

| | |
|---|---|
| Desfluoro impurity | 0.06 |
| Isomer impurity | 0.01 |
| Lactone impurity | 0.07 |

Tablets were subjected to in vitro dissolution testing using the USP Test 711 “Dissolution” procedure with type 2 apparatus, 75 rpm rotation, and 900 mL of either pH 6.8 phosphate buffer or 0.1 N hydrochloric acid as the medium, and compared with commercial LIPITOR 80 mg tablets. The results are shown in Table 2.

TABLE 2

| Cumulative % of Drug Dissolved | |
|---|---|
| Example 2 | LIPITOR ® |

| Minutes | Buffer | HCl | Buffer | HCl |
|---|---|---|---|---|
| 10 | 101 | 26 | 97 | 30 |
| 20 | 104 | 29 | 100 | 35 |
| 30 | 104 | 29 | 101 | 37 |
| 45 | 104 | 30 | 102 | 39 |
| 60 | 104 | 29 | 101 | 40 |

### EXAMPLE 3

### Bilayer Tablet Pharmaceutical Formulation of Atorvastatin 80 mg

| | |
|---|---|
| Ingredient | Grams |

LAYER I*

| | |
|---|---|
| Atorvastatin calcium-BHA premix (Example 1) | 261.803 |
| Lactose monohydrate | 792 |
| Prosolve ™ SMCC HD 90$$ | 1388.2 |
| Croscarmellose sodium | 108 |
| Sodium hydrogen carbonate | 51 |
| Hydroxypropylcellulose (Nisso) | 45 |
| Magnesium stearate | 18 |

LAYER II**
Intragranular

| | |
|---|---|
| Calcium carbonate (Fine grade) | 1584 |
| Croscarmellose sodium | 72 |
| Hydroxypropylcellulose (Klucel LF) # | 90 |
| Water*** | 1200 |

Extragranular

| | |
|---|---|
| Croscarmellose sodium | 90 |
| Magnesium stearate | 36 |

COATING^{&}

| | |
|---|---|
| Opadry White YS-1-7040^{$} | 72 |
| Isopropyl alcohol*** | 1260 |
| Dichloromethane*** | 90 |
| Water*** | 450 |

*Layer 1 prepared for 3,000 tablets.
**Layer 2 prepared for 6,000 tablets.
^{&}Coating dispersion prepared for 2,400 tablets.
***Evaporates during processing.
# Klucel ™ LF is a product of Aqualon.
^{$}Opadry White is a pre-formulated coating product of Colorcon Inc., containing hypromellose 5 cps, titanium dioxide, and macrogol/polyethylene glycol 400.
$$ Prosolv SMCC HD 90 is silicified microcrystalline cellulose and a product of JRS Pharma.

Manufacturing Process:

A. Layer I.

1) Atorvastatin calcium-BHA premix, sodium bicarbonate, Prosolv SMCC HD 90, lactose and croscarmellose sodium were sifted through an ASTM #40 mesh sieve and mixed in a blender for about 10 minutes.

2) Magnesium stearate was sifted through an ASTM #60 mesh sieve.

3) The sifted granules of step 1) were blended with magnesium stearate of step 2) for about 3 minutes.

B. Layer II.

1) Calcium carbonate and intragranular croscarmellose sodium were sifted through an ASTM #25 mesh sieve and dry mixed for about 10 minutes in a rapid mixer granulator.

2) Klucel LF was dissolved in water with stirring to form a granulating solution.

3) Step 1) materials were granulated using step 2) granulating solution.

4) The granules were dried at 60° C. for about 60 minutes in a fluid bed drier to obtain a loss on drying at 105 ° C. of 0.87% w/w.

5) Dried granules were sifted through an ASTM #20 mesh sieve.

6) Extragranular croscarmellose sodium was sifted through an ASTM #40 mesh sieve and magnesium stearate was sifted through an ASTM #60 mesh sieve.

7) The sifted granules of step 5) were blended with croscarmellose sodium of step 6) in a double cone blender for about 10 minutes.

8) The mixture of step 7) was blended with magnesium stearate of step 6) for about 5 minutes.

C. Compression.

1) Layer I blend (888 mg) and Layer II blend (312 mg) were compressed into bilayer tablets of 1200 mg average weight.

D. Coating.

1) A coating dispersion of Opadry OY-58900 was prepared using a mixture of isopropyl alcohol, water, and dichloromethane. The compressed tablets were coated to produce a 2% weight gain.

Tablets were subjected to in vitro dissolution testing in different dissolution media (pH 6.8 phosphate buffer and 0.1 N HCl) using the USP procedure with USP type 2 apparatus, 75 rpm rotation, and 900 mL of medium, and compared with the commercial product LIPITOR 80 mg tablets. The data are shown below.

Cumulative %
of Drug Dissolved

| Sample | Minutes | Buffer | 0.1N HCl |
|---|---|---|---|
| Example 3 | 10 | 84 | 25 |
| | 20 | 90 | 31 |
| | 30 | 91 | 34 |
| | 45 | 91 | 38 |
| LIPITOR | 10 | 97 | 29 |
| | 20 | 100 | 38 |
| | 30 | 101 | 43 |
| | 45 | 101 | 46 |

Tablets were evaluated in a two-way crossover pharmacokinetic study, involving administration of the 80 mg atorvastatin tablets of Example 3 as a test product (“T”) and the commercial product LIPITOR® 80 mg tablets as a reference product (“R”), with healthy human volunteers in the fasting state, and plasma concentrations of the drug compounds were determined at intervals after dosing.

The following parameters were calculated:

AUC₀₋ₜ=the area under plasma concentration versus time curve, from the time of administration to the last measurable concentration.

AUC_{0-∞}=area under the plasma concentration versus time curve, from the time of administration to infinity.

Cₘₐₓ=maximum plasma concentration.

The average pharmacokinetic parameters for each product were calculated and are summarized in the table below, where CI is the confidence interval.

| Parameter | AUC₀₋ₜ | AUC_{0-∞} | Cₘₐₓ |
|---|---|---|---|
| Ratio of mean Least | 105.106 | 105.001 | 88.825 |
| Square | | | |
| Values (T ÷ R) × 100 | | | |
| 90% CI (T ÷ R) × 100, | 90.53 | 92.12 | 70.17 |
| Lower Limit | | | |
| 90% CI (T ÷ R) × 100, | 122.04 | 119.68 | 112.46 |
| Upper Limit | | | |

### EXAMPLE 4

### Atorvastatin 80 mg Bilayer Tablet Pharmaceutical Formulation

| | |
|---|---|
| Ingredient | Quantity (in gms) |

LAYER I**
Intragranular

| | |
|---|---|
| Atorvastatin calcium | 165.46 |
| Lactose monohydrate (Impalpable | 528.54 |
| grade) | |
| Croscarmellose sodium (Ac-Di-Sol) | 48 |
| Sodium hydrogen carbonate | 60 |
| Hydroxypropylcellulose (Nisso-HPC-L) | 48 |
| Water* | 300 |

Extragranular

| | |
|---|---|
| Prosolve SMCC HD 90 | 818 |
| Croscarmellose sodium (Ac-Di-Sol) | 72 |
| Magnesium stearate | 24 |

LAYER II***

| | |
|---|---|
| Magnesium hydroxide (MS-90)$$ | 439.5 |
| Croscarmellose sodium (Ac-Di-Sol) | 35.25 |
| Magnesium stearate | 2.25 |

FILM COATING^{&}

| | |
|---|---|
| Opadry White OY-58900$ | 33.52 |
| Isopropyl alcohol* | 469.28 |
| Dichloromethane* | 33.52 |
| Water* | 167.6 |

**Layer I prepared for 2000 tablets.
***Layer II prepared for 1500 tablets.
^{&}Film coating dispersion prepared for 1200 tablets.
*Evaporates during processing.
$Opadry White is a pre-formulated coating product of Colorcon Inc., containing hypromellose 5 cps, titanium dioxide, and macrogol/polyethylene glycol 400.
$$Directly compressible grade magnesium hydroxide.

Manufacturing Process:

A. Layer I.

1) Atorvastatin calcium, sodium bicarbonate, lactose and intragranular croscarmellose sodium were sifted through an ASTM #30 mesh sieve and mixed in a rapid mixer granulator for about 10 minutes.

2) Hydroxypropylcellulose was dissolved in water with stirring to form a granulating solution.

3) Step 1) materials were granulated using step 2) granulating solution.

4) The granules were dried at 60° C. for 45 minutes in a fluid bed dryer to obtain a loss on drying at 105° C. less than 2% w/w.

5) Dried granules were sifted through an ASTM #20 mesh sieve.

6) Extragranular croscarmellose sodium and Prosolve SMCC HD 90 were sifted through an ASTM #20 mesh sieve and magnesium stearate was sifted through an ASTM #60 mesh sieve.

7) Sifted granules of step 5) were blended with croscarmellose sodium and Prosolve SMCC HD 90 of step 6) in a double cone blender for about 10 minutes.

8) The mixture of step 7) was blended with magnesium stearate of step 6) for about 5 minutes.

B. Layer II.

1) Magnesium hydroxide was sifted through an ASTM #20 mesh sieve.

2) Croscarmellose sodium was sifted through an ASTM #30 mesh sieve and magnesium stearate was sifted through an ASTM #60 mesh sieve.

3) Step 1 ingredient was blended with croscarmellose sodium of step 2) for about 10 minutes.

4) The mixture of step 3) was blended with magnesium stearate of step 2) for about 5 minutes.

C. Compression.

1) Layer I blend (882 mg) and Layer II (318 mg) blend were compressed into bilayer tablets of 1200 mg average weight.

D. Coating.

1) Coating dispersion of Opadry White OY-58900 was prepared using a mixture isopropyl alcohol, water, and dichloromethane. The compressed tablets were coated to produce a 2.3% weight gain.

Tablets were subjected to in vitro dissolution testing in various dissolution media (pH 6.8 phosphate buffer, 0.1 N HCl, and 0.001 N HCl) using the USP procedure with USP type 2 apparatus, 75 rpm rotation, and 900 mL of medium, and compared with the commercial product LIPITOR 80 mg tablets. The data are shown below.

Cumulative %
of Drug Dissolved

| Sample | Minutes | Buffer | 0.1N HCl | 0.001N HCl |
|---|---|---|---|---|
| Example 4 | 10 | 89 | 22 | 74 |
| | 20 | 93 | 29 | 95 |
| | 30 | 95 | 29 | 96 |
| | 45 | 98 | 29 | 100 |
| LIPITOR | 10 | 97 | 39 | 22 |
| | 20 | 100 | 46 | 63 |
| | 30 | 101 | 50 | 65 |
| | 45 | 101 | 52 | 66 |

Atorvastatin tablets were evaluated in a two-way crossover pharmacokinetic study, involving administration of the 80 mg atorvastatin tablets of Example 4 as a test product (“T”) and the commercial product LIPITOR 80 mg tablets as a reference product (“R”), with healthy human volunteers in the fasted state, and plasma concentrations of the drug compounds were determined at intervals after dosing.

The following parameters were calculated:

AUC₀₋ₜ=the area under plasma concentration versus time curve, from the time of administration to the last measurable concentration.

AUC_{0-∞}=area under the plasma concentration versus time curve, from the time of administration to infinity.

Cₘₐₓ=maximum plasma concentration.

The average pharmacokinetic parameters for each product were calculated and are summarized below, where CI is the confidence interval.

| Parameter | AUC₀₋ₜ | AUC_{0-∞} | Cₘₐₓ |
|---|---|---|---|
| Ratio of Mean Least Square Values | 109.779 | 109.724 | 102.409 |
| (T ÷ R) × 100 | | | |
| 90% CI (T ÷ R) × 100, Lower Limits | 98.66 | 98.66 | 82.71 |
| 90% CI (T ÷ R) × 100, Upper Limits | 122.16 | 122.01 | 126.79 |

### EXAMPLE 5

### Atorvastatin 80 mg Tablets with Alkalizing Agent and Surfactant

| | |
|---|---|
| | Quantity |
| Ingredient | (in kgs) |

Granulation

| | |
|---|---|
| Atorvastatin calcium-BHA pre-mix | 0.207 |
| (Example 1) | |
| Sodium lauryl sulphate | 0.1 |
| Sodium bicarbonate | 0.12 |
| Microcrystalline cellulose (Avicel PH112) | 1.281 |
| Crospovidone | 0.048 |
| Water‡ | 7 |

Extragranular

| | |
|---|---|
| Lactose monohydrate (Flowlac 100) | 0.181 |
| Microcrystalline cellulose (Avicel PH112) | 0.35 |
| Crospovidone | 0.06 |
| Hydroxypropylcellulose (Klucel LF) | 0.03 |
| Magnesium stearate | 0.024 |

Coating

| | |
|---|---|
| Opadry OY 58900 White | 0.048 |
| Isopropyl alcohol (IPA)‡ | 0.63 |
| Methylene chloride (MC)‡ | 0.046 |
| Water‡ | 0.23 |

‡Evaporates during processing.

Manufacturing Process:

Granulation.

1) Microcrystalline cellulose and crospovidone were sifted through an ASTM #30 mesh sieve and mixed for about 5 minutes.

2) Atorvastatin calcium-BHA pre-mix, sodium lauryl sulphate and sodium bicarbonate (previously sifted through an ASTM #40 mesh sieve) were dispersed in water with continuous stirring.

3) The mixture of step 1) was loaded into a fluid bed processor and the dispersion of step 2) was sprayed onto the mixture with a bed temperature of about 30-40° C.

4) The formed granules were dried in a fluid bed processor for another 20 minutes after spraying, until loss on drying was less than 2.5% w/w.

5) The dried granules were sifted through an ASTM #30 mesh sieve.

Extragranular Blending.

6) Microcrystalline cellulose, crospovidone, hydroxypropylcellulose, and lactose monohydrate were sifted through an ASTM #30 mesh sieve.

7) Magnesium stearate was sifted through an ASTM #60 mesh sieve.

8) The granules of step 5) and the sifted ingredients of step 6) were blended for 15 minutes in a double cone blender.

9) The step 8) mixture was blended with magnesium stearate of step 7) for about 5 minutes.

10) The lubricated blend of step 9) was compressed into tablets having an average weight of 960 mg.

Coating.

11) Opadry White OY 58900 was dispersed in a mixture of IPA, MC and water, and stirred for about 45 minutes.

12) The tablets of step 10) were coated, using dispersion of step 11).

### EXAMPLE 6

### Atorvastatin 80 mg Tablets with Surfactant and Acid Solubility-Enhancing Excipient

| | Kilograms | | | |
|---|---|---|---|---|
| Ingredient | 5A | 5B | 5C | 5D |

Part A Granulation

| | | | | |
|---|---|---|---|---|
| Atorvastatin calcium-BHA pre-mix | 0.179 | 0.033 | 0.025 | 0.017 |
| (Example 1) | | | | |
| Basic butylated methacrylate | 0.08 | 0.016 | 0.012 | 0.008 |
| copolymer (Eudragit ™ EPO) | | | | |
| Microcrystalline cellulose (Avicel | 4.144 | 0.829 | 0.833 | 0.836 |
| PH112) | | | | |
| Lactose monohydrate (Flowlac ™ 100) | 4.09 | 0.818 | 0.818 | 0.818 |
| Methanol‡ | 1.6 | 0.32 | 0.32 | 0.32 |

Part B Granulation

| | | | | |
|---|---|---|---|---|
| Atorvastatin calcium-BHA pre-mix | 0.662 | 0.132 | 0.141 | 0.149 |
| (Example 1) | | | | |
| Lactose monohydrate (Flowlac 100) | 1.258 | 0.26 | 0.257 | 0.252 |
| Sodium starch glycolate | 0.3 | — | — | — |
| Crospovidone | — | 0.06 | 0.06 | 0.06 |
| Sodium bicatbonate | 0.32 | 0.064 | 0.068 | 0.072 |
| Sodium lauryl sulphate | 0.4 | 0.08 | 0.08 | 0.08 |
| Water‡ | 0.7 | 0.14 | 0.14 | 0.14 |

Extragranular

| | | | | |
|---|---|---|---|---|
| Sodium starch glycolate | 0.36 | — | — | — |
| Crospovidone | — | 0.064 | 0.063 | 0.063 |
| Hydroxypropylcellulose (Klucel LF) | 0.16 | 0.032 | 0.032 | 0.032 |
| Magnesium stearate | 0.12 | 0.024 | 0.024 | 0.024 |

Coating

| | | | | |
|---|---|---|---|---|
| Opadry OY-58900 White | 0.354 | 0.048 | 0.048 | 0.048 |
| Isopropyl alcohol (IPA)‡ | 4.7 | 0.63 | 0.63 | 0.63 |
| Methylene chloride (MC)‡ | 0.34 | 0.046 | 0.046 | 0.046 |
| Water‡ | 1.68 | 0.23 | 0.23 | 0.23 |

‡Evaporates during processing.

Manufacturing Process:

Part A Granulation.

1) Microcrystalline cellulose and lactose monohydrate were sifted through an ASTM #40 mesh sieve and mixed for 5 minutes.

2) Atorvastatin calcium-BHA pre-mix and Eudragit EPO (previously sifted through an ASTM #40 mesh sieve) were dissolved in methanol.

3) The blend of step 1) was loaded into a fluid bed processor bowl and the solution of step 2) was top sprayed with a product temperature of about 30-40° C.

4) The formed granules were dried in the fluid bed processor for 20 minutes until loss on drying at 105° C. was less than 3% w/w.

5) The dried granules sifted through an ASTM #30 mesh sieve.

Part B Granulation.

6) Atorvastatin calcium-BHA pre-mix, lactose monohydrate, crospovidone, sodium starch glycolate, and sodium bicarbonate were sifted through an ASTM #30 mesh sieve and mixed for about 10 minutes in a granulator.

7) Sodium lauryl sulfate was dispersed in water and the mixture of step

6) was granulated using sodium lauryl sulphate dispersion.

8) The wet granules were dried in a fluid bed drier at 60° C. until loss on drying at 105° C. was less than 3% w/w.

9) The dried granules were sifted through an ASTM #30 mesh sieve.

Extragranular Blending.

10) Crospovidone, sodium starch glycolate, and hydroxypropylcellulose were sifted through an ASTM #20 mesh sieve.

11) Magnesium stearate was sifted through an ASTM #60 mesh sieve.

12) The granules of step 9) and the sifted ingredients of step 10) were blended for 10 minutes in double cone blender.

13) The mixture of step 12) was blended with magnesium stearate of step 11) for 5 minutes.

14) The lubricated blend of step 13) was compressed into tablets having an average weight of 1206 mg.

Coating.

15) Opadry White OY 58900 was dispersed in a mixture of IPA, MC and water, and stirred for about 45 minutes.

16) The tablets of step 14) were coated, using dispersion of step 15).

### EXAMPLE 7

### Atorvastatin 80 mg Tablets with Surfactant and Acid Solubility-Enhancing Excipient

| | |
|---|---|
| Ingredient | Kilograms |

Granulation A

| | |
|---|---|
| Atorvastatin calcium-BHA pre-mix | 12.42 |
| (Example 1)* | |
| Basic butylated methacrylate copolymer | 6 |
| (Eudragit EPO) | |
| Microcrystalline cellulose (Avicel PH112) | 416.58 |
| Lactose monohydrate (compressible grade) | 409 |
| Methanol‡ | q.s. |

Granulation B

| | |
|---|---|
| Atorvastatin calcium-BHA pre-mix* | 70.31 |
| Lactose monohydrate (compressible grade) | 128.69 |
| Sodium bicarbonate | 34 |
| Sodium lauryl sulphate (Texapon K12P) | 40 |
| Water‡ | q.s. |

Extragranular

| | |
|---|---|
| Crospovidone | 12 |
| Hydroxypropylcellulose (Nisso HPC-L) | 64 |
| Magnesium stearate | 12 |

Coating

| | |
|---|---|
| Opadry White AMB OY-B-28920 | 25 |
| Water‡ | q.s. |

*82.73 mg of pre-mix is equivalent to 80 mg of atorvastatin.
‡Evaporates during processing.

### Manufacturing Process:

Granulation A.

1) Sift microcrystalline cellulose and lactose monohydrate through a #40 mesh sieve and mix for 5 minutes.

2) Dissolve atorvastatin calcium-BHA pre-mix and Eudragit EPO (previously sifted through a #40 mesh sieve) in methanol.

3) Load the blend of step 1) into a fluid bed processor bowl and spray the solution of step 2) using a top spray configuration, with a bed temperature of about 30°-40° C.

4) Dry the formed granules in the fluid bed processor after spraying, until the loss on drying at 105° C. is less than 3% w/w.

5) Sift the dried granules through a #30 mesh sieve.

Granulation B.

6) Sift atorvastatin calcium-BHA pre-mix, lactose monohydrate, and sodium bicarbonate through a #30 mesh sieve and mix for 10 minutes.

7) Disperse sodium lauryl sulfate in water to form a paste and granulate the mixture of step 6) in a rapid mixer granulator.

8) Dry the wet granules in a fluid bed dryer at 60° C. until a loss on drying at 105° C. is less than 3% w/w.

9) Sift the dried granules through a #30 mesh sieve, mill any retained particles through a 1 mm screen, then pass milled particles through the sieve and combine with the first sieved granules.

Extragranular Blending.

10) Sift crospovidone and hydroxypropylcellulose through a #20 mesh sieve.

11) Sift magnesium stearate through a #60 mesh sieve.

12) Blend the granules of steps 5) and 9) with the sifted ingredients of step 10) for 10 minutes in a double cone blender.

13) Blend the step 12) mixture with magnesium stearate of step 11) for 5 minutes in the double cone blender.

Compression.

14) Compress the mixture of step 13) into tablets having an average weight of 1205 mg.

Coating.

15) Coat the tablets using a dispersion of Opadry White in water, to produce a weight gain of about 1.25% w/w.

Properties of Uncoated Tablets:

| Parameter | Result |
|---|---|
| Average weight (10 tablets) | 1210.5 |
| Hardness range (Kp) | 17.0-18.5 |
| Thickness range (mm) | 7.43-7.47 |
| Friability (% w/w) | Nil |
| Disintegration Time, in water | 10-13 minutes |

Coated tablets were subjected to in vitro dissolution testing in different dissolution media (pH 6.8 phosphate buffer, 0.1 N HCl, and 0.001 N HCl) using the USP procedure with USP type 2 apparatus, 75 rpm rotation, and 900 mL of medium, and were compared with the reference product LIPITOR 80 mg tablets. The data are given below:

Cumulative %
of Drug Dissolved

| Sample | Minutes | Buffer | 0.1N HCl | 0.001N HCl |
|---|---|---|---|---|
| Example 7 | 10 | 43 | 27 | 25 |
| | 20 | 81 | 30 | 48 |
| | 30 | 94 | 30 | 59 |
| | 45 | 97 | 29 | 60 |
| LIPITOR | 10 | 97 | 39 | 22 |
| | 20 | 100 | 46 | 63 |
| | 30 | 101 | 50 | 65 |
| | 45 | 101 | 52 | 66 |

A two-way crossover pharmacokinetic study was conducted, involving administration of the 80 mg atorvastatin tablets of Example 7 as a test product (“T”) and the commercial product LIPITOR® 80 mg tablets as a reference product (“R”), with healthy human volunteers in fasted and fed states, and plasma concentrations of the drug compounds were determined at intervals after dosing.

The following parameters were calculated:

AUC₀₋ₜ=the area under plasma concentration versus time curve, from the time of administration to the last measurable concentration.

AUC_{0-∞}=area under the plasma concentration versus time curve, from the time of administration to infinity.

Cₘₐₓ=maximum plasma concentration.

The average pharmacokinetic parameters for each product were calculated and are summarized in the following tables, where CI is a confidence interval.

### Fasting Study

| Parameter | AUC₀₋ₜ | AUC_{0-∞} | Cₘₐₓ |
|---|---|---|---|
| Ratio of Mean Least Square | 110.51 | 110.04 | 106.22 |
| Values (T ÷ R) × 100 | | | |
| 90% CI (T ÷ R) × 100, Lower Limit | 100.49 | 100.29 | 86.97 |
| 90% CI (T ÷ R) × 100, Upper Limit | 121.54 | 120.73 | 129.73 |

### Fed Study

| Parameter | AUC₀₋ₜ | AUC_{0-∞} | Cₘₐₓ |
|---|---|---|---|
| Ratio of Mean Least Square | 99.74 | 100.27 | 88.47 |
| Values (T ÷ R) × 100 | | | |
| 90% CI (T ÷ R) × 100, Lower Limit | 91.42 | 92.24 | 74.99 |
| 90% CI (T ÷ R) × 100, Upper Limit | 108.82 | 109.00 | 104.38 |

### EXAMPLE 8

### Atorvastatin 80 mg Tablets with Alkalizer

| | |
|---|---|
| Ingredient | mg/Tablet |

Granulation

| | |
|---|---|
| Atorvastatin calcium-BHA premix (Example 1) | 82.73 |
| Sodium carbonate USP | 30 |
| Mannitol USP | 344.77 |
| Sodium hydroxide USP | 6.5 |
| Water* | q.s. |

Extragranular

| | |
|---|---|
| Microcrystalline cellulose NF (Avicel PH102) | 200 |
| Mannitol USP | 188 |
| Sodium bicarbonate NF | 264 |
| Crospovidone NF | 60 |
| Magnesium stearate NF | 24 |

Film Coating

| | |
|---|---|
| Opadry White AMB OY-B-28920 | 30 |
| Water* | q.s. |

*Evaporates during processing.

Manufacturing Procedure:

1. Atorvastatin calcium, sodium carbonate and mannitol were sifted through a #30 mesh sieve.

2. Sodium hydroxide was dissolved in water.

3. Step 2 solution was used to granulate step 1 blend in a rapid mixer granulator.

4. Granules were dried in a fluid bed dryer at 60° C., until loss on drying at 105° C. was less than 3% w/w, and then were sifted through a #40 mesh sieve.

5. Extragranular sodium bicarbonate, mannitol, microcrystalline cellulose (MCC) and crospovidone were sifted through a #40 mesh sieve.

6. Magnesium stearate was sifted through a #60 mesh sieve.

7. Granules of step 4 were blended with the step 5 mixture and then blended with step 6 magnesium stearate.

8. Step 7 blend was compressed into tablets and coated using Opadry White AMB OY-B-28920 dispersion in water.

The tablets were packaged in aluminum foil blisters and stored under accelerated stability testing conditions (40° C. and 75% relative humidity) for 3 months. Samples were analyzed at intervals for degradation product content, and results are shown below, where values are percentages of the label atorvastatin content.

| Impurity | Initial | 1 Month | 2 Months | 3 Months |
|---|---|---|---|---|
| Desflouro | 0.043 | 0.053 | 0.060 | 0.058 |
| Isomer | BLD* | 0.008 | 0.018 | 0.007 |
| Lactone | 0.012 | 0.011 | 0.017 | 0.027 |
| Total | 0.407 | 0.845 | 0.684 | 0.808 |

*BLD = Below the limit of detection.

1. A pharmaceutical formulation comprising:
a) atorvastatin, or a pharmaceutically acceptable salt thereof;
b) at least one pharmaceutically acceptable surfactant;
c) at least one pharmaceutically acceptable acid solubility-enhancing excipient; and
d) optionally, at least one pharmaceutically acceptable excipient.

2. The pharmaceutical formulation of claim 1, wherein an acid solubility-enhancing agent comprises one or more pharmaceutically acceptable polymers.

3. The pharmaceutical formulation of claim 1, wherein an acid solubility-enhancing agent is a cationic copolymer based on dimethylaminoethyl methacrylate and neutral methacrylates.

4. A pharmaceutical formulation comprising:
a) atorvastatin, or a pharmaceutically acceptable salt thereof;
b) at least one pharmaceutically acceptable surfactant;
c) at least one pharmaceutically acceptable alkalizing agent; and
d) optionally, at least one pharmaceutically acceptable excipient.

5. The pharmaceutical formulation of claim 4, wherein a surfactant is ionic.

6. The pharmaceutical formulation of claim 4, wherein a surfactant comprises sodium lauryl sulfate.

7. The pharmaceutical formulation of claim 4, wherein an alkalizing agent comprises one or more of a sodium, potassium, magnesium, or calcium citrate, carbonate, bicarbonate, phosphate, sulfate, sulfite, benzoate, or ascorbate salt.

8. The pharmaceutical formulation of claim 4, which is a tablet dosage form.

9. The pharmaceutical formulation of claim 4, which is a capsule dosage form.

10. A bilayer pharmaceutical formulation comprising:
atorvastatin, or a pharmaceutically acceptable salt thereof, in a first layer, optionally together with one or more pharmaceutically acceptable excipients; and at least one pharmaceutically acceptable alkaline compound in a second layer, optionally together with one or more pharmaceutically acceptable excipients.

11. The bilayer pharmaceutical formulation of claim 10, wherein an alkaline compound comprises an alkali metal salt, an alkaline earth metal salt, an organic alkaline compound, or any mixtures thereof.

12. The bilayer pharmaceutical formulation of claim 10, wherein an alkaline compound comprises one or more of a magnesium or calcium citrate, carbonate, bicarbonate, phosphate, sulfate, sulfite, benzoate, or ascorbate salt.

13. The bilayer pharmaceutical formulation of claim 10, wherein an alkaline compound comprises magnesium hydroxide, calcium carbonate, or a mixture thereof.

14. A pharmaceutical formulation comprising a solubility-enhanced form of atorvastatin or a pharmaceutically acceptable salt thereof, together with at least one pharmaceutically acceptable excipient.

15. The pharmaceutical formulation of claim 14, wherein a solubility-enhanced form of atorvastatin, or a pharmaceutically acceptable salt thereof, is in the form of micronized particles or nanoparticles.

16. The pharmaceutical formulation of claim 14, comprising atorvastatin calcium in the form of particles having D₉₀ less than about 1000 nm.

17. The pharmaceutical formulation of claim 14, comprising atorvastatin calcium in the form of particles having D₉₀ less than about 500 nm.

18. The pharmaceutical formulation of claim 14, comprising atorvastatin calcium in the form of particles having D₅₀ less than about 500 nm.

19. The pharmaceutical formulation of claim 14, comprising atorvastatin calcium in the form of particles having D₅₀ less than about 250 nm.

20. The pharmaceutical formulation of claim 1, wherein a surfactant is ionic.

21. The pharmaceutical formulation of claim 1, wherein a surfactant comprises sodium lauryl sulfate.

22. The pharmaceutical formulation of claim 1, which is a tablet dosage form.

23. The pharmaceutical formulation of claim 1, which is a capsule dosage form.
